(19) United States
(12) Patent Application Publication
(10) Pub. No.: US 2021/0023119 A1
(43) Pub. Date: Jan. 28, 2021

(54) METHODS FOR THE TREATMENT OF PROSTATE CANCER

(71) Applicant: Cleveland State University, Cleveland, OH (US)

(72) Inventors: Girish C. Shukla, Cleveland, OH (US); Jey Sabith Ebron, Cleveland, OH (US)

(21) Appl. No.: 16/937,007

(22) Filed: Jul. 23, 2020

Related U.S. Application Data
(60) Provisional application No. 62/877,576, filed on Jul. 23, 2019.

(51) Int. Cl. A61K 31/7105; A61P 35/00; C12N 15/113; A61K 45/06

(52) U.S. Cl. CPC A61K 31/7105; A61K 45/06; C12N 15/1136; A61P 35/00

(57) ABSTRACT

The general inventive concepts contemplate methods and compositions for reducing or treating therapeutic resistance associated with prostate cancer in an individual in need thereof. In certain exemplary embodiments, the method comprises administration of miR-644a or a composition that increases a level of miR-644a in an individual.

### CROSS-REFERENCE TO RELATED APPLICATIONS

This application claims priority to and any benefit of U.S. Provisional Patent Application No. 62/877,576, filed Jul. 23, 2019, the content of which is hereby incorporated herein as if recited in its entirety.

### FIELD

The general inventive concepts relate to the field of medical therapies and more particularly to methods for the treatment or prevention of prostate cancer.

### BACKGROUND

Prostate cancer (PCa) is the most common non-cutaneous cancer affecting men in the USA and ranks second in cancer-related deaths. PCa carcinogenesis and metastasis encompass significant genetic heterogeneity. Numerous alternations in prostate cells lead to PCa progression from a castration-sensitive to a castration-resistant PCa (CRPC) which relates to poor prognosis for patients. However, the molecular mechanisms contributing to the development of CRPC are only beginning to emerge. Clinical and scientific evidence indicates that CRPC contains clones of both Androgen Receptor (AR)-negative and AR-positive tumor cells. AR is a ligand-dependent transcription factor, and androgen signaling plays a crucial homeostatic role in balancing the normal physiology and biology of the prostate. Paradoxically, AR and the androgen signaling axis is also a predominant causative factor in the development of PCa and its transition to castration-resistant cancer.

The current therapeutic options for PCa include Androgen Deprivation Therapy (ADT) and AR inhibitors. However, the development of resistance to these drugs leads to a poor prognosis for CRPC patients. Interestingly, large-scale integrative genomic and proteomic analyses of CRPC tumors reveal that multiple intrinsic signaling pathways also play a causative role in the development of CRPC. Hence, an in-depth understanding of the molecular and genetic changes that occur during the development of CRPC is necessary to identify novel therapeutic targets. Considering the molecular complexity of the AR signaling cascade and PCa heterogeneity, it is logical to co-target the expression of genes involved in multiple carcinogenic pathways to achieve a sustained and clinically significant response.

Regulatory noncoding microRNAs (miRNAs) fine-tune posttranscriptional control of gene expression and thus regulatory networks. miRNAs regulate gene expression by guiding the association between the RNA-induced silencing complex (RISC) and target messenger RNAs (mRNA). Numerous miRNAs regulate expression of genes involved in cell cycle, organogenesis, energy balance and development thus affecting cell proliferation, differentiation, stem cell maintenance, and cell death, as well as many other cellular functions. Depending upon the differential expression of miRNAs in a given cell and tissue, miRNAs either promote oncogenesis (oncomiRs) or act as tumor suppressors. In the context of prostate carcinogenesis, multiple intrinsic mechanisms, including androgen signaling, AR spliced variants, steroid metabolism, Myelocytomatosis viral oncogene homolog (c-Myc) aberrant transcriptional activation miRNA dysregulation are significant factors in the development of CRPC. The inherent property of miRNAs to fine-tune gene expression by suppressing the translation of multiple genes of signaling networks involved in disease-promoting activities makes them attractive natural molecules in miRNA replacement therapy.

### SUMMARY

A need exists for treatment options for individuals having prostate cancer, including those who experience castration-resistant prostate cancer after androgen deprivation therapy.

The general inventive concepts are based on a new understanding of PCa progress and resistance. The general inventive concepts provide a powerful tool for modulation of PCa progression and, more particularly, the general inventive concepts are directed to compositions and methods for reduction or interruption of certain biological processes associated with prostate cancer, such methods and compositions comprise miR-644a, and its use. In particular, miR-644a has been shown to modulate the expression of Bcl-x1 and/or Bcl-2. In certain exemplary embodiments, the methods and compositions may comprise an additional miR, including miR149-5p.

In certain embodiments, the general inventive concepts are directed to a method of reducing or treating therapeutic resistance associated with prostate cancer in an individual in need thereof. In certain exemplary embodiments, the method comprises administration of miR-644a or a composition comprising miR-644a.

In an exemplary embodiment, the general inventive concepts contemplate a composition comprising miR-644a. In certain exemplary embodiments, the composition may comprise an additional miR, including miR149-5p.

In an exemplary embodiment, the general inventive concepts contemplate a vector for introducing miR-644a for the treatment or prevention of prostate cancer. In certain exemplary embodiments, the vector may introduce an additional miR, including miR149-5p.

The general inventive concepts are based, in part, on recognition of the importance of influencing the tumor microenvironment by miRNA to potentiate therapeutic relief mediated by androgen-signaling inhibitor Enzalutamide. miR-644a suppresses the expression of AR expression and many essential genes involved in cell metabolism, proliferation, and EMT which promotes CRPC. The general inventive concepts and examples described herein demonstrate the profound anti-tumor function of miR-644a by inhibiting the expression of multiple cellular pathways that drive tumor progression and microenvironment.

Numerous other aspects, advantages, and/or features of the general inventive concepts will become more readily apparent from the following detailed description of exemplary embodiments and from the accompanying drawings being submitted herewith.

### BRIEF DESCRIPTION OF THE DRAWINGS

The general inventive concepts, as well as embodiments and advantages thereof, are described below in greater detail, by way of example, with reference to the drawings in which:

FIG. 1A shows immunoblot analysis of AR gene expression in PCa cell lines LNCaP, LAPC4 and 22RV1 treated with miR-644a at concentrations 20 nM, 50 nM and 100 nM compared with lipofectamine treated cells (Mock) and cells treated with negative control mimic (NC). 22RV1 cells express both the full length AR (110 KDa) as well as the alternatively spliced isoform lacking the ligand binding domain (80 KDa). The signal intensities of bands were measured using the IMAGEJ image analysis software. The AR expression in each lane was determined by normalizing AR band (110 KDa) intensity to hsp70 band intensity.

FIG. 1B shows RT-qPCR analysis of AR mRNA expression in LNCaP, LAPC4 and 22RV1 cells transfected with miR-644a or NC mimic. Each bar represents AR mRNA expression normalized to 18S rRNA expression. Data are plotted as mean±SE of three independent experiments. Asterisks indicate statistical significance as determined by the independent samples t-test (**p<0.01, ***p<0.001).

FIG. 1C shows a schematic representation of firefly luciferase reporter construct containing 244 nucleotide sequence from AR 3′ UTR with either WT or mutant (MUT) miR-644a target site. In the MUT-UTR construct, 8 nucleotides in the seed matching region of the target site were mutated to their complementary nucleotides to disrupt miR-644a binding.

FIG. 1D shows a luciferase reporter assay in CHO-K1 cells co-transfected with WT-UTR or MUT-UTR constructs and miR-644a mimic or NC mimics as described in methods.

FIG. 2A shows RT-qPCR analysis of AR co-regulators mRNA expression in LNCaP cells transfected with miR-644a or NC mimic in the presence of DHT (20 nM). Each bar represents the corresponding mRNA expression normalized to 18S rRNA expression. Data are plotted as mean±SE of three independent experiments. Asterisks indicate statistical significance as determined by the independent samples t-test (*p<0.05, **p<0.01 and ***p<0.001).

FIG. 2B shows immunoblot analysis of AR co-regulators protein expression in PCa cell lines LNCaP and PC-3 cells treated with miR-644a or NC mimics.

FIG. 2C shows an analysis of secreted PSA in cell culture supernatants using PSA Sandwich ELISA and RT-qPCR analysis of PSA mRNA expression in LNCaP and 22RV1 cells transfected with miR-644a or NC mimic, in the presence of DHT. Each bar represents PSA mRNA expression normalized to 18S rRNA expression. Data are plotted as mean±SE of three independent experiments. Asterisks indicate statistical significance as determined by the independent samples t-test. *p<0.05 between miR-644a treated samples and NC.

FIG. 3A shows a representative images of 22RV1 xenografts in NCR nu/nu mice subcutaneously injected with 2×10⁶ 22RV1 cells on both flanks. After 10 days of injection, tumors were treated using miR-644a, Enzalutamide, and the combination of miR-644a along with Enzalutamide and the control animals without treatment.

FIGS. 3B and 3C show that tumor xenograft size was measured every three days from the initial signs of tumor development to plot tumor growth curves. The arrow in the graph represents the start of treatment. Animals were sacrificed on day 32 after injection and tumors excised. miR-644a treated mice showed significant tumor growth inhibition compared to Enzalutamide treated as well as untreated mice. Data are plotted as mean values±SEM (n=5), *p<0.05. Representative images of excised tumors from mice treated with miR-644a and the controls.

FIG. 3D shows expression values of significant genes downregulated during miR-644a treatment in 22RV1 xenografts from NGS dataset represented as a heat map, shown in red and blue shades relative to the mean expression values of the genes in linear scale. The heat map shows downregulated potential targets genes of miR-644a in tumor xenografts.

FIG. 4A shows soft agar colony formation assay in 22RV1 cells treated with miR-644a and negative control (NC) mimic and quantification of colony numbers per field measured as a mean value of multiple measurements.

FIG. 4B shows results of a cell viability assay in LNCaP and PC-3 cells treated with miR-644a and NC mimics. Data are plotted as mean±SE of three independent experiments. Independent samples t-test was used to assess statistical significance. Asterisks indicate a significant difference from NC mimics transfected cells (**p<0.01).

FIG. 4C shows immunoblot analysis of total and cleaved PARP levels in LNCaP and PC-3 cells indicating induction of apoptosis.

FIG. 4D shows cell death detection Elisa assay to detect apoptosis in LNCaP cells treated with miR-644a mimic compared with NC mimic and lipofectamine treated cells as well as cells treated with Enzalutamide (ENZ).

FIG. 4E shows immunoblot analysis of Bcl-x1 and Bcl-2 gene expression in PCa cell lines LNCaP and PC-3 treated with miR-644a and NC mimics.

FIG. 4F shows qRT-PCR analysis of Bcl-2 and Bcl-x1 mRNA expression in LNCaP cells transfected with miR-644a or NC mimics. Each bar represents the relative gene expression normalized to 18S rRNA expression. Data are plotted as mean±SE of three independent experiments. Asterisks indicate statistical significance as determined by the independent samples t-test (*p<0.05, **p<0.01).

FIG. 5A shows immunoblot analysis of c-Myc, IGF-1R and AKT gene expression in PCa cell lines.

FIG. 5B shows qRT-PCR analysis of c-Myc mRNA expression in LNCaP cells overexpressing miR-644a mimic. Each bar represents corresponding gene mRNA expression normalized to 18S rRNA expression. Data are plotted as mean±SE of three independent experiments. Asterisks indicate statistical significance as determined by the independent samples t-test (*p<0.05, **p<0.01).

FIGS. 5C and 5D show measurement of extracellular acidification rate (ECAR) was assessed in real time by XF24 flux analyzer in (C) 22RV1 and (D) PC-3 cells. The cells were deprived of glucose for 2 hrs before the assay followed by Glycolytic stress test by the addition of Glucose (10 mM), oligomycin (1 μM) and 2-deoxyglucose (50 mM). ECAR was measured under basal conditions after transfection with mir-644a (20 nM). Data points represent group means±SE, n=3.

FIG. 6A shows transwell migration assay using matrigel coated membranes in 22RV1 cells treated with miR-644a compared to NC mimic and quantitation of transwell assay by measuring a mean number of cells per field comparing miR-644a treated cells with NC mimic treated cells.

FIG. 6B is an immunoblot analysis of EMT inducers ZEB1, cdk6 and snail as well as the immunoblot for EMT markers E-Cadherin, β-catenin, and vimentin gene expression in PC-3 cells treated with miR-644a at concentrations (20 nM) compared with cells treated with NC mimic.

FIG. 6C shows qRT-PCR analysis of mesenchymal markers (Vimentin, Snail, and ZEB1) and Epithelial markers (CLDN4 and E-Cadherin) mRNA expression in PC-3 cells transfected with miR-644a or NC mimics. Each bar represents the corresponding gene mRNA expression normalized to 18S mRNA expression.

FIG. 6D shows a wound healing assay in LNCaP and PC-3 cells after treatment with miR-644a or NC mimics. The quantitation of cell covered area in miR-644a transfected cells was analyzed using Wimasis image analysis software. Data are plotted as mean±SE of three independent experiments. Asterisks indicate statistical significance as determined by the independent samples t-test (**p<0.01, ***p<0.001).

FIG. 7A shows in situ hybridization of a tissue section with miR-644a probe compared with U6 snRNA as a control. Increased AR levels are observed between samples from patients who underwent ADT compared to no ADT patients, magnification ×400. Quantitation of in situ hybridization data measuring the counts per field which displays the inverse correlation between miR-644a and AR gene expression.

FIG. 7B shows miR-644a expression analysis in RNA extracted from patient tissue samples by RT-qPCR. Normal samples correspond to benign regions of the patients, n=10. G (3+3) corresponds to patient samples with a Gleason score of (3+3), n=10. G (4+3) corresponds to patient samples with a Gleason score (4+3), n=10. Metastasis samples correspond to samples from multiple sites of metastasis including lymph node, liver and adrenal metastasis (n=10). Data are plotted as mean±SE of three independent experiments. Asterisks indicate statistical significance as determined by the independent samples t-test (*p<0.05, **p<0.01).

### DETAILED DESCRIPTION

While the general inventive concepts are susceptible of embodiment in many different forms, there are shown in the drawings, and will be described herein in detail, specific embodiments thereof with the understanding that the present disclosure is to be considered an exemplification of the principles of the general inventive concepts. Accordingly, the general inventive concepts are not intended to be limited to the specific embodiments illustrated herein.

The materials, systems, and methods described herein are intended to be used to provide compositions and methods for the treatment and/or prevention of prostate cancer.

Castration-resistant prostate cancer (CRPC) is defined by tumor microenvironment heterogeneity affecting intrinsic cellular mechanisms including dysregulated androgen signaling, aerobic glycolysis (Warburg effect), and aberrant activation of transcription factors including androgen receptor (AR) and c-Myc. Using in vitro, in vivo and animal models applicants discovered a direct correlation between miR-644a downregulation and dysregulation of essential cellular processes. miR-644a downregulated expression of diverse tumor microenvironment drivers including c-Myc, AR co-regulators and anti-apoptosis factors Bcl-x1 and Bcl-2. Moreover, miR-644a modulates epithelial-mesenchymal transition (EMT) by directly targeting EMT promoting factors ZEB1, cdk6, and Snail. Finally, miR-644a expression suppresses the Warburg effect by direct targeting of c-Myc, Akt, IGF-1R and GAPDH expression. RNA-seq analysis revealed an analogous downregulation of these factors in animal tumor xenografts. This data demonstrates miR-644a mediated fine-tuning of oncogenic stimulating pathways and resultant potentiation of Enzalutamide therapy in CRPC patients.

Clinical genomics research has developed a better understanding of the genetic alterations in CRPC in recent years. However, translational application of these findings to develop more durable and effective treatments of CRPC is significantly hampered by the inability to identify functionally and clinically relevant drivers of CRPC and to develop an effective common control of their fine-tuned expression for homeostatic cellular controls. Besides, development of drug resistance in CRPC driven by enhanced invasion as well as metastasis remains a significant challenge in the clinical management and treatment of the disease. The critical deterrent in PCa diagnosis and prognosis is the molecular and clonal heterogeneity of the disease, which leads to major concerns of over treatment of indolent tumors and undertreatment of high-risk disease, leading to the development of metastatic disease. Due to the inherent heterogeneous nature of PCa involving pathways including androgen signaling, PI3K/AKT, Wnt/β-Catenin, EMT, AR coactivators, c-Myc and intratumoral steroidogenesis, developing a common therapeutic to target or at least fine-tune the expression of multiple cellular pathways has not been attempted in the clinical setting due to apparent reasons. Indeed, the development of next-generation androgen signaling inhibitors including Enzalutamide and Abiraterone acetate has been the most noticeable advancement in PCa therapeutics. In advanced prostate tumors, however, this therapy fails in the majority of patients and drug resistance is a daunting challenge.

The general inventive concepts are based, in part, on the discovery that a miRNA, miR-644a, that can fine-tune the expression of major causative pathways including androgen signaling, c-Myc, EMT, tumor energy metabolism via glycolysis and potentiate the therapeutic effects of CRPC drug Enzalutamide. An earlier study has demonstrated that miR-644a could target the expression of AR in LNCaP cells by measuring the protein levels only. A recent report confirmed the role of miR-644a in suppressing drug resistance by inhibition of cell survival and EMT in breast cancer complementing our findings. Another study recently demonstrated that miR-644a inhibits gastric cancer cell proliferation and invasion as well as promotes apoptosis in liver cancer cells.

Applicants' in-silico analysis revealed implication of miR-644a target pool in multiple cancer-related signaling pathways including metabolism, cell cycle, apoptosis, and signal transduction. In particular, clinically relevant oncogenes including c-Myc, SRC and metabolic oncogenes FASN and GLS. Other notable targets of miR-644a include AKT, β-catenin and SRC kinase, which collectively plays a significant role in metastatic PCa disease.

In addition, many co-activators which alter the transcriptional activity of AR including cdk6, SRC1, SRC2, SRC3, CBP, CCND1, and ARA24 have been implicated in PCa. In particular, targeting the expression of p160 group of coactivators SRC1, SRC2, and SRC3 by miR-644a would not only reduce the activation of the AR but also interfere with the ligand-independent activation of AR by IGF-1 and Akt pathways. Further, cdk6 interacts with AR and enhances its transcriptional activity in PCa. CBP is part of the p300-CBP complex, which is a transcriptional integrator that modulates a large number of transcription factors. Elevated expression of CBP has been associated with androgen ablation and involved in proliferation and agonistic activity of anti-androgens. miR-644a not only targets AR and c-Myc expression in PCa cells, but it also fine-tunes the expression of AR co-activators offering an advantage over conventional AR-targeted therapies.

Elevated expression of Bcl-2 and Bcl-x1 and the resulting dysregulation of apoptosis pathway is linked to CRPC and poor prognosis leading to metastasis. The role of miR-644a in enhancing apoptosis in both castration-sensitive and -resistant cells is strengthened by validating anti-apoptotic genes Bcl-2 and Bcl-x1 as its direct targets in addition to c-Myc.

MiR-644a may modulate cell proliferation by regulating critical effectors in cell cycle including c-Myc, cdk6 and CCND1. In addition to targeting CCND1 a regulator of cell proliferation, miR-644a also downregulates cdk6 which is a binding partner of CCND1. Furthermore, cdk6 plays a significant role in AR signaling by activating AR and is implicated in cell cycle progression. Elevated expression of cdk6 in androgen-sensitive PCa cells in response to androgen is involved in cell cycle and enhanced AR activity.

Most importantly, we have revealed a novel role of miR-644a in EMT, a process that plays a crucial role in the development of metastatic CRPC. EMT in PCa is mediated by activation of Snail and ZEB-1 by AR, which leads to repression of E-Cadherin and increased expression of 3-catenin. cdk6 is elevated in PCa cells and correlates with tumor grade, and further cdk6 interacts with AR and enhances AR activity. Likewise, both ZEB1 and Snail expression is upregulated in PCa and are implicated in invasion and metastasis. By targeting Snail, ZEB-1, SRC-1 and cdk6 together, miR-644a leads to downregulation of EMT markers including 3-catenin, Vimentin and increased expression of upregulation of E-Cadherin. Applicants' results validate the effect of miR-644a on EMT in both androgen-sensitive and castration-resistant cells. Taken together, miR-644a may modulate EMT in PCa cells in an AR-independent manner by targeting the critical regulators in PCa.

While not wishing to be bound by theory, miR-644a may modify the cell's proteome and thus alter its phenotype and tumor growth potential. miR-644a targets and downregulates factors involved in tumor energy metabolism of PCa cells including GAPDH, a glycolytic enzyme implicated in tumor metabolism and proto-oncogene c-Myc and overexpression of c-Myc contributes to the genesis of many human cancers including PCa. Growth factor-mediated expression initiates signaling via c-Myc and PI3K/AKT/mTOR pathways, and c-Myc promotes the expression of genes involved in amino acid transport and protein synthesis, therefore unbalancing the energy homeostasis, in favor of tumor growth. Interestingly, aerobic glycolysis flux through GAPDH is the rate-limiting step in the pathway and control points in glycolysis, play an essential role in tumor metabolism. Applicants demonstrate that miR-644a targets c-Myc, and GAPDH directly and downregulates the glycolysis and glycolytic capacity in PCa cells.

These results show that miR-644a directly regulates expression of various CRPC relevant targets including AR, AR coactivators, Bcl-2, Bcl-x1, c-Myc, EMT drivers including Snail, cdk6, and ZEB1. Instead of targeting the function of one oncogenesis-promoting factor by the drug, e.g., AR targeting by AR-antagonist, it would be potentially beneficial to fine-tune the expression of multiple pathways by miRNA replacement adjunctive therapy and to potentiate therapeutic effects of androgen signaling inhibitors Enzalutamide and Abiraterone acetate. By in vivo, in vitro, and animal experiments it has been shown that a large number of genes are post-transcriptionally regulated by miR-644a either directly or indirectly. Specifically, experiments in our animal model using 22RV1 cells containing AR-V7 show that miR-644a sensitizes the tumors to Enzalutamide treatment. Although, marginal effects are shown with miR-644a, a synergistic effect is shown through targeting both isoforms of AR as well as the numerous oncogenesis signaling pathways which might be beneficial for patients with advanced disease.

miR-644a anti-survival and anti-proliferation biological properties appear to play a vital role in androgen signaling, apoptosis, and tumor energy metabolic pathways. Applicants demonstrate that miR-644a inhibits or rather fine-tunes the expression of the molecular pathways, which promote tumor energy metabolism and Warburg effect, as well as genes that are drivers of CRPC development of metastasis, in a coordinated manner support the strong potential of miRNA therapeutic.

Accordingly, the general inventive concepts relate to new understanding of PCa progress and resistance. The general inventive concepts provide a powerful tool for modulation of PCa progression and, more particularly, the general inventive concepts are directed to compositions and methods for reduction or interruption of certain biological processes associated with prostate cancer, such methods and compositions comprise miR-644a, and its use. In particular, miR-644a has been shown to modulate the expression of Bcl-x1 and/or Bcl-2. In certain exemplary embodiments, the methods and compositions may comprise an additional miR, including miR149-5p.

In an exemplary embodiment, the general inventive concepts contemplate a method for providing gene therapy to an individual in need of prostate cancer therapy.

In certain exemplary embodiments, the general inventive concepts are directed to an expression vector comprising a first nucleic acid molecule. In an exemplary embodiment, the general inventive concepts contemplate a vector for introducing at least one microRNA molecule.

In certain exemplary embodiments, the expression vector comprises miR-644a or a nucleic acid molecule encoding for miR-644a. In certain exemplary embodiments, the expression vector comprises an additional miR, including miR149-5p.

### EXAMPLES

The following examples describe various compositions and methods for the modulation of various biological processes involved in prostate cancer progression and associated therapeutic resistance.

In-silico prediction of miR-644a as a master regulator of molecular pathways implicated in PCa. By computational prediction, applicants identified a handful of miRNAs that have the potential to target the AR 3′UTR (FIG. S1A). Next, applicants tested if these predicted miRNAs can negatively modulate the posttranscriptional AR expression in PCa cells. Applicants transfected a panel of predicted AR-targeting miRNA mimics in LNCaP and C4-2B cells and found that miR-644a maximally reduced AR expression as compared to other predicted AR-targeting miRNA mimics (Lane 8, FIGS. S1B and C). Earlier applicants have shown that miR-644a targets β-actin and Glyceraldehyde-3-Phosphate Dehydrogenase (GAPDH) gene expression, two proteins critical to cytoskeletal dynamics and glycolysis, respectively. To test the prospect of miR-644a to regulate posttranscriptional expression of multiple other genes involved in disease-promoting activities, Applicants in silico determined the putative targets of miR-644a potentially associated with prostate carcinogenesis promoting pathways. Applicants used miRecords, which predicts miRNA targets by considering at least four prominent algorithms including Targetscan 6.2, miRanda, miRtarget2 and RNAhybrid. miR-644a predicted target genes were subjected to KEGG and Gene Ontology pathways analysis by comparing to gene set comprehensive libraries from multiple sources including KEGG, Wikipathways, BioCarta, and Reactome.

Additionally, expression data analysis from 498 adenocarcinoma patients from Cosmic V78 revealed that miR-644a is predicted to target genes involved in pathways regulating apoptosis and hypoxia as well as NOTCH, c-Myc and androgen signaling pathways (Supplemental Table 1).

Furthermore, our bioinformatics analysis also predicted a potential role of miR-644a in other cancers including small cell lung cancer, pancreatic cancer, chronic myeloid leukemia and colorectal cancers (Supplemental Table 1). The bioinformatics analysis of experimentally validated gene expression datasets including EnrichR, Reactome and Cytoscape, predicted many genes including AR, Bcl-2, Bcl-x1, c-Myc, SRC, Snail, EZH2, E2F1 and ZEB1 as miR-644a targets (supplemental table 2). These genes have also been associated with PCa and development of resistance to androgen signaling inhibitors including Enzalutamide and Abiraterone acetate.

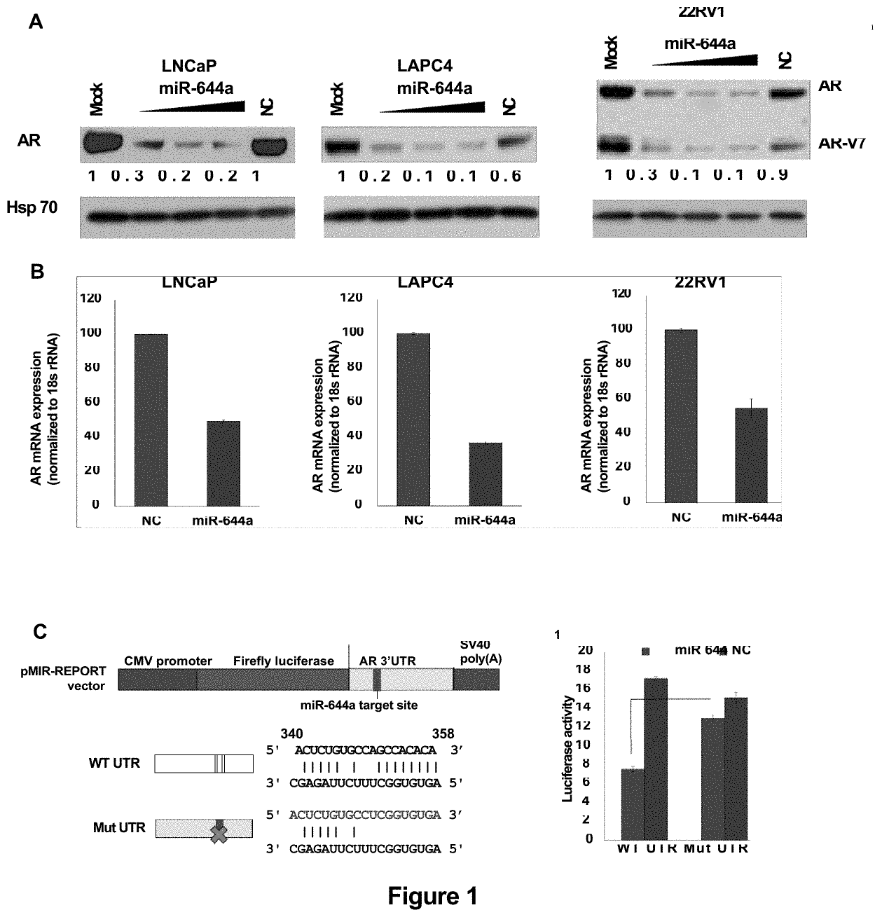

miR-644a downregulates AR expression and transactivation in PCa cells. To characterize miR-644a mediated regulation of AR expression and transactivation function, we ectopically overexpressed miR-644a mimics in multiple AR-positive PCa cell lines, including LNCaP, LAPC4, and 22RV1 cell lines express both AR mRNA and AR protein and represent castration-sensitive and -resistant cell models. Ectopic expression of miR-644a mimics in LNCaP and LAPC4 cells significantly reduced AR protein levels as compared to mock and negative control (NC) mimic transfected cells. Similarly, reduction in both full length and AR-V7 protein levels by miR-644a was also detected in 22RV1 cells (FIG. 1A), supporting the potential of miR-644a in targeting both isoforms of AR implicated in the development of drug resistance and CRPC. Hsp70 expression was used as a loading control since it is not a predicted target of miR-644a. Furthermore, RT-qPCR experiments confirmed significant downregulation of AR mRNA in miR-644a transfected LNCaP, LAPC4 and 22RV1 cells (FIG. 1B). These results demonstrate that miR-644a effectively suppresses AR expression in both castration-sensitive and -resistant cellular models of PCa. Furthermore, miRNA:mRNA firefly luciferase target validation experiments confirm a direct miR-644a interaction with the 3′ UTR of AR from nucleotides 340 to 358 (FIGS. 1C and D). The above data indicate that miR-644a targets AR gene expression and reduces both mRNA and protein levels in PCa cells by directly interacting with the 3′UTR of AR mRNA.

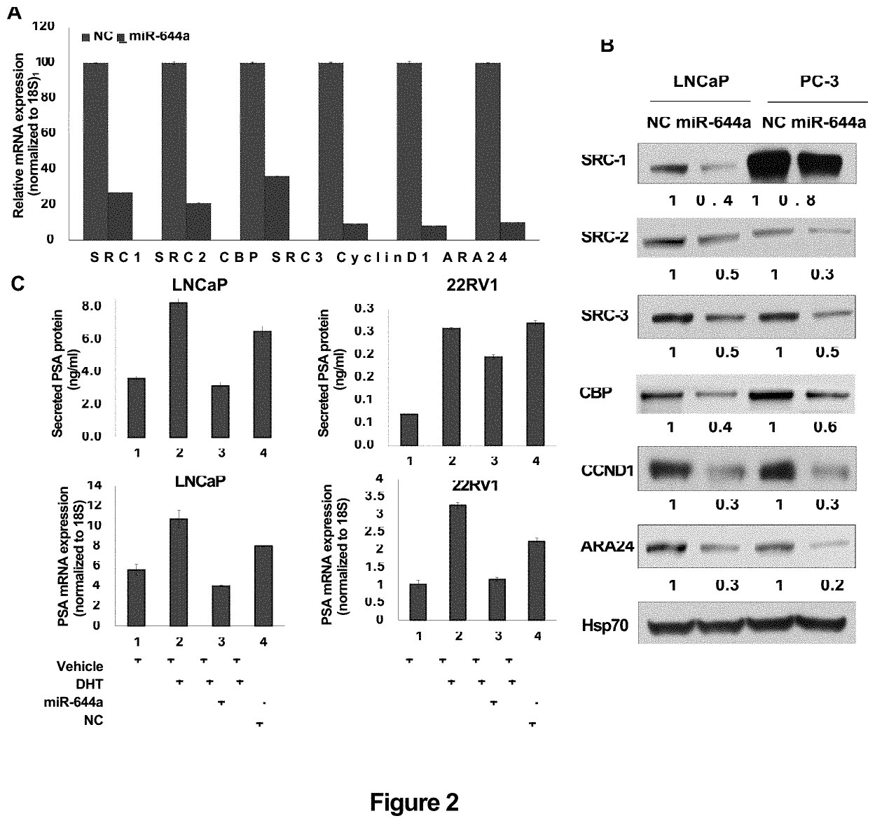

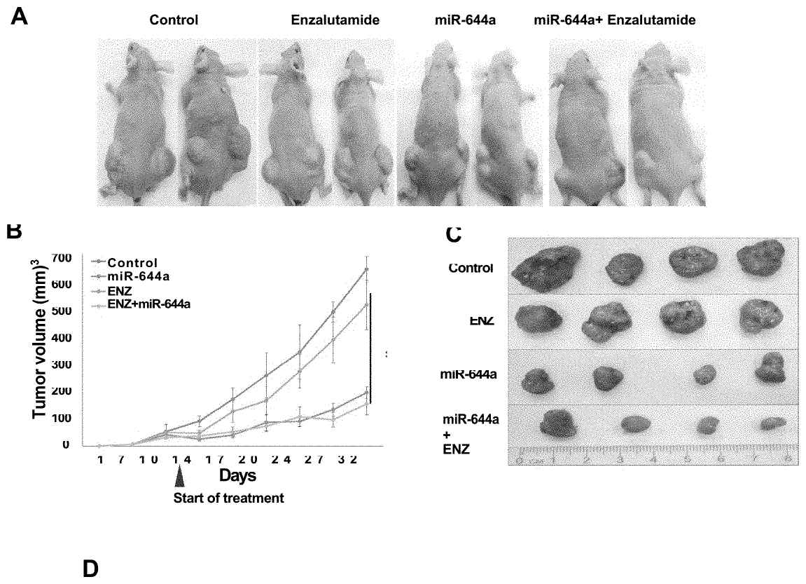

miR-644a downregulates AR transactivation function by suppressing the expression of AR co-regulators. Relapsed AR expression and its transactivation function is a key event in prostate carcinogenesis and a major cause of castration-resistant disease. AR co-regulator proteins play an important regulatory role in AR transcriptional activities, and many of these co-regulators are highly upregulated in CRPC (27). Several AR co-regulators were predicted targets of miR-644a, suggesting a fine-tuning paradigm (Supplemental Table 2). Among AR co-regulators, steroid receptor coactivator-1 (SRC1), steroid receptor coactivator-2 (SRC2), CREB-binding protein (CBP), steroid receptor coactivator-3 (SRC3), CyclinD1 (CCND1) and AR associated protein-24 (ARA24) were predicted targets of miR-644a (Supplemental Table 4). Next, we validated the expression of predicted AR co-regulators by RT-qPCR and immunoblotting in miR-644a overexpressing PCa cells. miR-644a over-expression resulted in significant downregulation of the expression of AR co-regulators including SRC-1, SRC-2, SRC-3, CCND1, CBP and ARA24 at mRNA level (FIG. 2A) as well as the protein level in LNCaP and PC-3 cells (FIG. 2B). The AR null PC-3 cell line is an established model to study androgen signaling-independent miR-644a biological activities. Additionally, we confirmed the inhibitory function of miR-644a on AR transactivation and signaling by analyzing the PSA protein and mRNA levels in AR-positive LNCaP and 22RV1 cells (FIG. 2C). Above data demonstrate that by the direct interaction with AR and its co-activators miR-644a contributes to the negative regulation of AR transactivation activities.

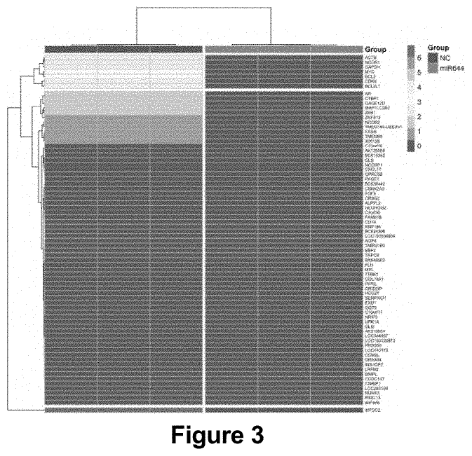

miR-644a is a potent tumor suppressor. To evaluate plausible tumor suppressor potential of miR-644a, we subcutaneously injected 22RV1 cells in NCR nu/nu mice on both flanks (n=5) and treated mice with intratumoral injections of miR-644a mimic. We compared treatment groups that received Enzalutamide through oral gavage and the group that received a combination of miR-644a with Enzalutamide. Tumor volumes of miR-644a injected mice were significantly lower than those receiving Enzalutamide alone demonstrating potentiation of Enzalutamide therapy by miR-644a (FIGS. 3A, B and C). Intratumoral delivery of miR-644a reduced tumor growth by >50% compared to that of NC mimics. We confirmed the expression of miR-644a by RT-qPCR in harvested tumors to determine the delivery and expression of mimics (FIG. S2A). Immunoblotting of tumor lysates confirmed that miR-644a treatment correlates with downregulation of AR WT (110 kDa) as well as AR-V7 (80 kDa) isoforms (FIG. S2B). We also validated the AR transactivation function by PSA expression analysis of serum collected from the animals (FIG. S2C). Furthermore, mRNAseq data obtained from the tumors show several genes were downregulated in miR-644a treated animals (FIG. 3D). Functional analysis of the downregulated genes using KEGG indicates that miR-644a is predicted to target genes involved in proliferation, apoptosis, EMT and aerobic glycolysis. Many of these genes are also associated with multiple cancer pathways supporting a tumor suppressor role of miR-644a (Supplemental Table 5). AR, SRC-1, SRC-1, GAPDH, ACTB among many others were downregulated in tumors instilling the confidence in the mRNASeq dataset for further exploration of other downregulated genes' as potential targets of miR-644a. In summary, the xenograft experiment determined a potent tumor suppressor function of miR-644a, presumably by fine-tuning the expression of androgen signaling-dependent as well as -independent cellular pathways.

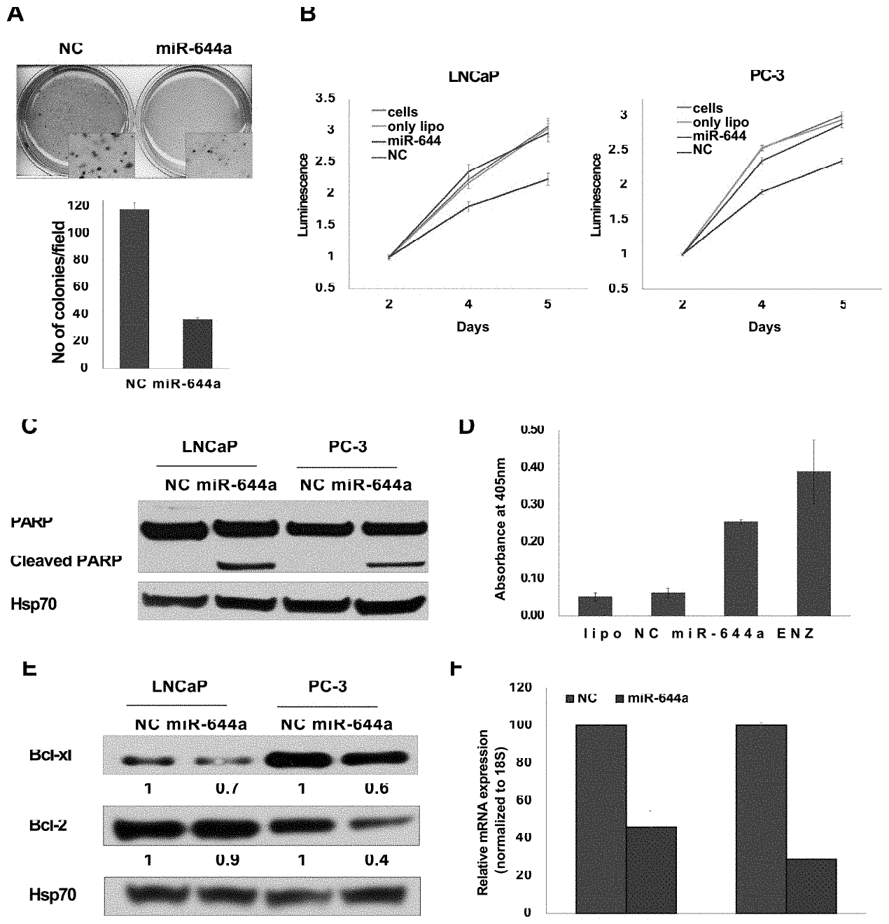

miR-644a expression in PCa cells promotes pro-apoptotic activities. Next, we determined the functional significance of miR-644a expression in the growth and viability of PCa cells. The clonogenic soft agar assay in 22RV1 cells demonstrated that the overexpression of miR-644a affected growth and survival of PCa cells as apparent by a decrease in the number of colonies as well as the decrease in the size of the colonies compared to the NC mimic (FIG. 4A). Also, cell viability assays demonstrate a significant growth inhibition by miR-644a in both, LNCaP and PC-3 cells (FIG. 4B). We reasoned that the growth inhibitory effect of miR-644a on cancer cells could be attributed to its pro-apoptotic activities. Hence, we determined PARP (poly ADP ribose polymerase) cleavage and downregulation of anti-apoptotic biomarkers in miR-644a overexpressing LNCaP and PC-3 cells. Indeed, in miR-644a transfected cells, PARP proteolytic cleavage was prominent when compared to NC transfected cells as determined by immunoblotting (FIG. 4C). We further verified the pro-apoptosis activity of miR-644a by using a quantitative sandwich immunoassay, which demonstrated that miR-644a expressing cells have higher levels of apoptosis as compared to NC mimic transfected cells, as well as AR antagonist Enzalutamide treated cells (FIG. 4D). Moreover, the level of apoptosis in the miR-644a transfected cells approaches the level of apoptosis induced by treatment with Enzalutamide.

Further, characterization of miR-644a treated cells for apoptotic biomarkers using a protein microarray revealed miR-644a induced changes consistent with apoptosis in LNCaP and PC3 cells (FIG. S3). Multiple pro-apoptotic post-translational modifications of proteins including cleaved caspase-3, cleaved caspase-7, Bad Ser136, SAPK/JNK Thr183/Tyr185, cleaved PARP, and p53 Ser15 were upregulated. Additionally, anti-apoptotic markers AKT Ser473 phosphorylation and Survivin were downregulated (FIG. S3 and supplemental table 3). We further confirmed that the pro-apoptotic function of miR-644a is mediated by the direct targeting of two anti-apoptotic genes, Bcl-x1 and Bcl-2 (FIGS. 4E and F) in PCa cells.

The effect of miR-644a transfection on the proliferation of PCa cells was also analyzed by BrdU incorporation in miR-644a treated LNCaP and PC-3 cells. miR-644a substantially decreased proliferation in both cell lines (FIGS. S4A and B), indicating a significant role of miR-644a in regulating growth, survival, and proliferation of PCa cells. Collectively, these results demonstrate that miR-644a can suppress the growth and proliferation of PCa cells.

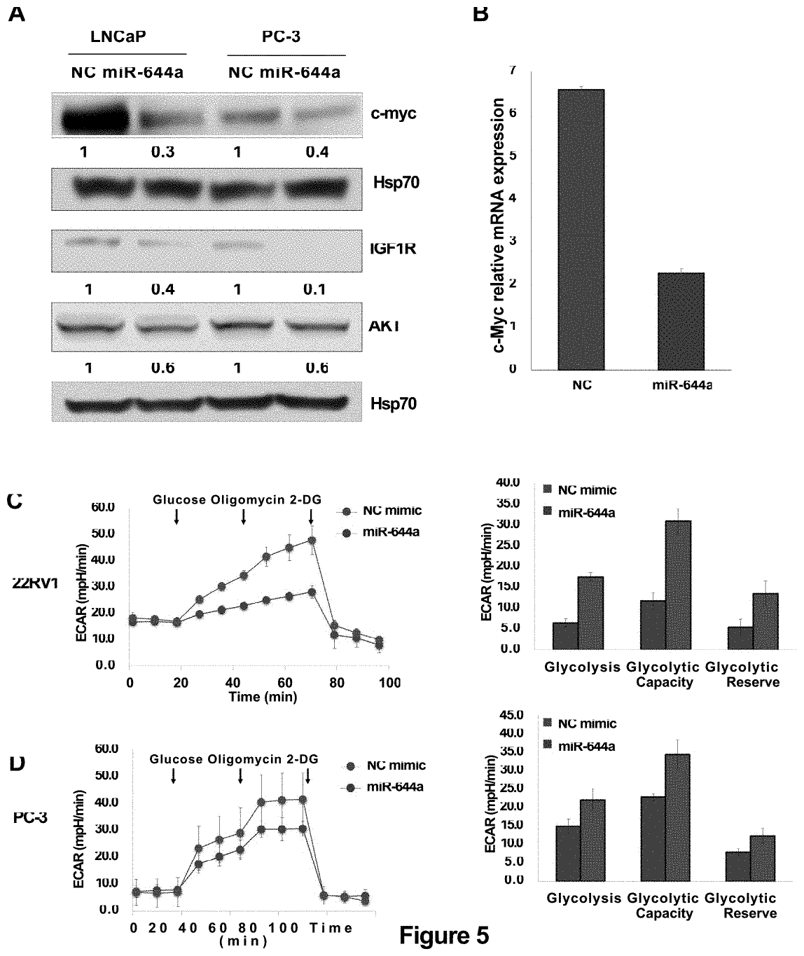

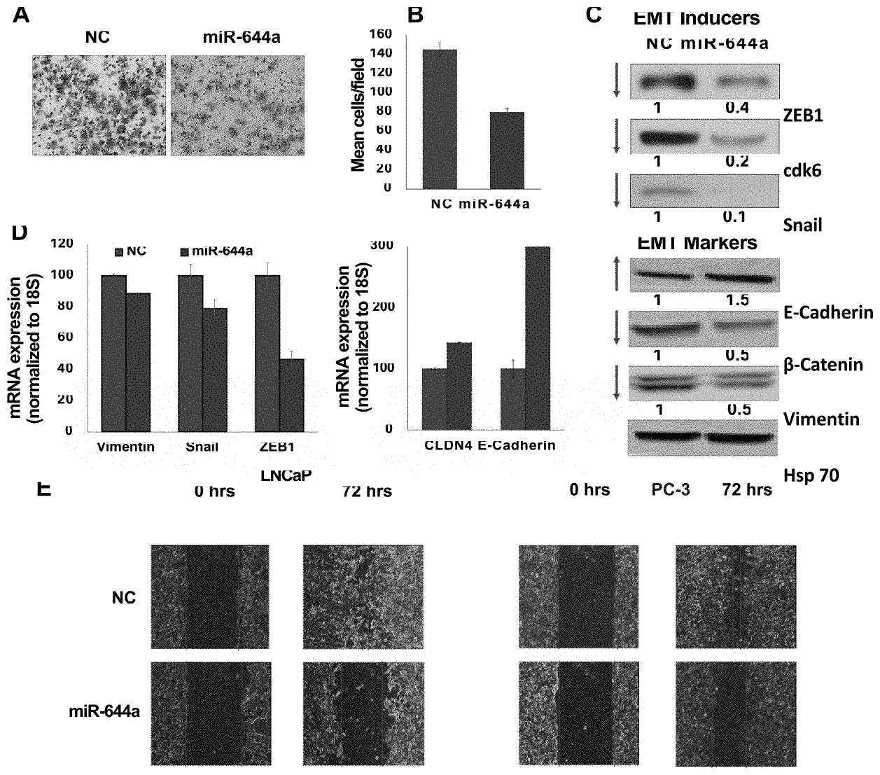

miR-644a suppresses the Warburg effect in PCa cells and tumors. To further delineate the underlying mechanism for the anti-tumor activity of miR-644a, we sought to determine the expression of known key genes implicated in PCa tumor metabolism. The cellular pathway analysis predicted the proto-oncogene c-Myc as a potential target of miR-644a, among many others; hence, we performed c-Myc gene expression analysis. Immunoblots and RT-qPCR determined the downregulation of c-Myc both, at protein and mRNA levels, respectively in miR-644a overexpressing cells (FIGS. 5A and B).

Further, Applicants confirmed that c-Myc is a direct target of miR-644a using a firefly luciferase reporter. Overexpression of miR-644a mimics repressed firefly luciferase expression containing c-Myc 3′UTR, whereas the seed region mutations in the miR-644a target sequence rescued the repression of luciferase (FIG. S5B). Above experiments determined, that miR-644a mediated downregulation of c-Myc expression may be of therapeutic significance in castration-resistant disease.

Additionally, AKT and IGF-1R contribute to aerobic glycolysis and tumor growth in PCa (29). Essentially, Insulin-like growth factor (IGF-1) signaling is mediated by the activation of Insulin-like growth factor receptor (IGF-1R), which promotes AR signaling in CRPC via activation of AR-V7 splice variant. Increased IGF-1R levels in PCa cells are associated with the development and progression of the disease and a valuble target in CRPC treated with IGF-1R antagonists (31). Since IGF-1R/AKT signaling contributes to tumor energy metabolism and castration-resistant AR signaling, we validated the expression of these factors in miR-644a overexpressing PCa cells. Immunoblotting of both LNCaP and PC3 cell lysates revealed downregulation of IGF-1R and AKT expression in both castration-sensitive and -resistant cell lines (FIG. 5A).

Tumor energy metabolism is mainly dependent on the glycolytic pathway rather than oxidative phosphorylation, and GAPDH is a central player in glycolysis-dependent energy supply forming the core of cancer cell survival. Upregulated expression of GAPDH in high-grade tumors underlines the importance of metabolism in PCa (32). Since GAPDH expression is downregulated by miR-644a by direct interaction we examined its enzymatic activity in PCa cells as well as in the xenografts. The downregulated GAPDH activity in both 22RV1 xenograft tumors as well as in PC-3 cells (FIGS. S5C & D), indicates miR-644a antagonist role in tumor energy metabolism. Further glycolytic extracellular flux analysis revealed that miR-644a significantly decreased glycolysis and glycolytic capacity in 22RV1 and PC-3 cells (FIGS. 5C & D). Collective repression of c-Myc, Akt, IGF-1R and GAPDH expression by miR-644a that suppresses Warburg effect and proliferation underlines that miR-644a is a potent prostate tumor suppressor.

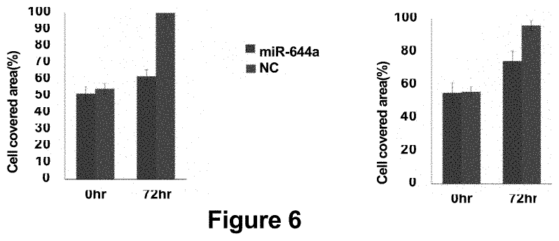

miR-644a targets the expression of critical genes which stimulate epithelial-mesenchymal transition in PCa. Our in-silico analysis also predicted EMT genes as potential targets of miR-644a, including Zinc Finger E-Box Binding Homeobox 1 (ZEB1), cyclin-dependent kinase 6 (cdk6) and Snail family zinc finger 1 (snail). Therefore, we analyzed if miR-644a exerts any influence on EMT in PCa cells. Our results indicated that miR-644a suppresses invasion of 22RV1 (FIGS. 6A & B) as well as PC-3 cells (data not shown). Such repression of EMT phenotype is likely to be mediated via the targeting of ZEB1, cdk6, and snail in PCa. We confirmed the downregulation of ZEB1, cdk6, and snail, both at protein and mRNA levels in miR-644a overexpressing cells (FIGS. 6C and D). Furthermore, Firefly luciferase reporter target assays determined that ZEB1, cdk6, and snail genes are direct targets of miR-644a (FIGS. S6A, B & C). Also, the reversal of EMT was confirmed by immunoblotting for EMT markers Vimentin, E-Cadherin and β-catenin in PC-3 cells (FIG. 6C). Wound healing assay in LNCaP and PC-3 cells overexpressing miR-644a confirmed the inhibition of migration of PCa cells (FIG. 6E). Together, the above data highlight the significance of fine-tuning of the expression of multiple regulators of EMT by miR-644a for improved PCa therapeutics.

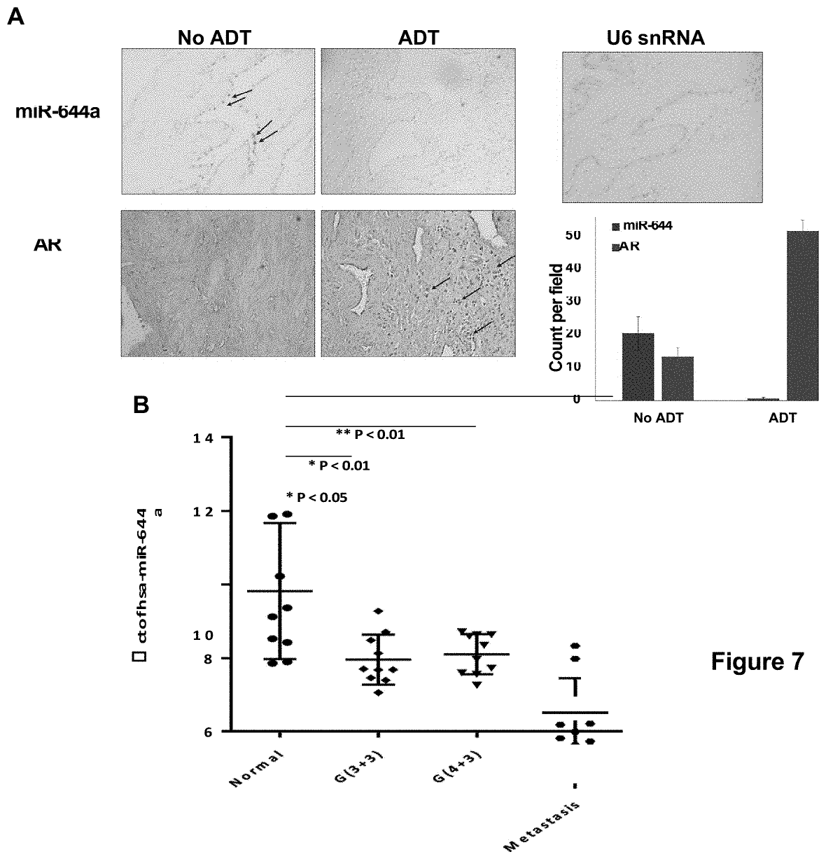

miR-644a target genes correlate with copy number variation of ITCH and more aggressive tumors. Using the cBioPortal (PMID 23550210, PMID: 22588877) to access TCGA prostate cohorts we examined the expression and copy number variation (CNV) of the miR-644a host gene, ITCH. Strikingly ITCH was amplified in the neuroendocrine cohort of tumors (PMID: 26855148) (Supplemental FIG. 7A). We, therefore, examined the correlation of expression of the miR-644a target genes with the CNV of ITCH in the NEPC cohort. The cross-correlation plots (Supplemental FIG. 7B) reveal that in the NEPC cohort there was a significant negative correlation between ITCH amplification and the expression of 11 miR-644a target genes including NCOR2.

It was also reasoned that in tumor cohorts even when clear amplification of ITCH was not apparent, miR-644a targets may be associated with the disease that is more aggressive. For example, we examined the expression of the most altered miR-644a targets in the PRAD cohort (PMID: 26544944). By filtering these genes to those that were most altered in expression, tumors were clustered into two groups (Supplemental FIG. 7C). A chi-squared test established that this grouping of tumor significantly stratified patients by whether they experienced tumor recurrence or not.

miR-644a is a potential prognostic drug resistance biomarker. To determine the differential expression of mature miR-644a, we analyzed its expression in PCa tissue derived from patients with or without ADT. miR-644a expression is downregulated in patients after ADT treatment and is inversely correlated with AR expression in the same tissue (FIG. 7A). This inverse correlation implicates the regulation of AR gene expression by miR-644a. Further, comparisons of patient samples from different stages of PCa and benign tissues revealed that miR-644a expression was highly downregulated in metastatic PCa samples (FIG. 7B), supporting a tumor suppressor role for miR-644a in PCa. This observation was also validated in PCa cell lines demonstrating loss of miR-644a expression in multiple PCa cell lines compared to benign epithelial cells (FIG. S8).

Methods: Human PCa cell lines (LNCaP, DU-145, 22RV1 and PC-3 with catalog numbers CRL-1740, HTB-81, CRL-2505, and CRL-1435 respectively) were purchased from ATCC (Manassas, Va.) and C4.2B was purchased from Viromed Laboratories (Minnetonka, Minn.). The cells were cultured in complete RPMI 1640 medium supplemented with 10% fetal bovine serum (FBS), 2 mM L-glutamine, penicillin and streptomycin antibiotics. LNCaP cells were used to mimic the model of hormone dependency in PCa. DU-145 and PC-3 cells were utilized as models of castration resistant disease. Further, Applicants utilized 22RV1 cells that express an alternatively spliced AR isoformAR-V7 to test the therapeutic potential of miR-644a during therapeutic resistance. CHO-K1 cells were cultured in Dulbecco's Modified Eagle's Medium (DMEM) supplemented with 5% FBS, 2 mM L-glutamine, 1 mM L-proline, 10 mM 4-(2-Hydroxyethyl)piperazine-1-ethanesulfonic acid (HEPES) and antibiotics. All the cell lines were maintained in a humidified 5% CO2 atmosphere at 37° C. All the experiments were repeated at least three times with two stocks of cells.

Construction of reporter plasmids, transfections and luciferase assays. The target validation of miR-644a target sites was performed as described previously (21). In brief, WT UTR (WT: wild type) reporter plasmids were constructed by cloning fragments of 3′ UTR spanning the predicted target site for miR-644 downstream of the firefly luciferase coding region in pMIR-REPORT vector (Ambion, Austin, Tex.). The primers used to amplify the 3′UTR fragments, and the fragment sizes are provided in the Supplemental Table 4. Site-directed mutagenesis of the putative target site for miR-644 in WT UTR construct was carried out to generate the MUT UTR constructs. Nucleotide sequences of the constructs were confirmed by DNA sequencing. For luciferase assays, CHO-K1 cells (30,000 cells/well) were plated in 24-well plates one day before transfection. For all miRNA target validation experiments, cells were transfected using Lipofectamine 2000 (Invitrogen), with 100 ng of WT UTR or MUT UTR firefly luciferase reporter construct, 0.5 ng of Renilla luciferase reporter plasmid (Promega, Madison, Wis.) and either miR-644 mimic (10 nM) or NC mimic (10 nM). Cell lysates were assayed for firefly and Renilla luciferase activities 48 hr after transfection using the Dual-Luciferase Reporter Assay System (Promega) in Victor 3 Multilabel Counter 1420 (PerkinElmer). Renilla luciferase activity served as a control for transfection efficiency. Data are represented as a ratio of firefly luciferase activity to Renilla luciferase activity.

Quantitative real-time PCR analysis of miR-644a expression. Total RNA from tissue samples with varying disease severity and normal tissues were obtained from Prostate Cancer Biorepository Network. First strand cDNA was synthesized from 100 ng of total RNA from PCa cells or patient samples using stem-loop primers specific for human mature miR-644 and snoRNA (RNU66). Reverse transcription and quantitative real-time (qRT)-PCR was carried out using the TaqMan MicroRNA Reverse Transcription kit and TaqMan MicroRNA Assays (Applied Biosystems, Foster City, Calif.) as described previously. The relative expression of miR-644a was calculated as 2^{-ΔCt} where ΔCt=Ct value of miR-644a in a sample—Ct value of RNU66 in that sample. Mean miR-expression±standard error (SE) was calculated from three independent experiments.

Immunoblotting to determine protein levels in miR-644a overexpressing cells. In this study, to detect and quantify the expression of proteins in all PCa cells we transiently transfected the cells with miR-644a mimic or NC mimic in 50 nM concentration or as indicated for a particular experiment. For apoptosis detection, in addition to miR-644a treatments, the LNCaP cells were or treated with a known AR antagonist, Bicalutamide (100 μM) and apoptosis was assayed 4 days post-transfection. Apoptotic was determined by using the cell death detection ELISA^{PLUS} kit (Roche Applied Science, Indianapolis, Ind.) according to the manufacturer's protocols.

Confocal Microscopy. PCa cells were seeded in six-well plates at 200,000 cells per well with coverslip one day before transfection. After 48 hours of transfection, the slides were fixed using ice-cold methanol for 5 minutes followed by permeabilization using 0.25% Triton X in 1X PBS. The slides were blocked for 1 hour in 1% BSA in 1X PBS and stained for antibodies. The secondary antibodies used were the Alexa Fluor 488 and 647 conjugated secondary antibodies (1:200). Nuclei were stained by incubating the slides in DAPI (1:10000) for five minutes at room temperature followed by washes with 1X PBS. The prepared slides were visualized using a Nikon A1RSI confocal microscope.

Drug treatment: PCa cells were seeded in six-well plates at 200,000 cells per well 48 hrs before transfection. The cells were maintained in charcoal/dextran stripped FBS and transfected with the miR-644 mimics using Lipofectamine 3000 reagent. The cells were treated with Enzalutamide at the concentration of 1.0 μM. After 24 hrs of treatment with miR-mimics and Enzalutamide, the cells were induced for AR activation using DHT (10 nM) or DMSO as a control. 24 hrs after DHT induction, the cells were processed for further analysis.

Tumor xenograft experiments: 22RV1 human prostate cell xenografts were established in male athymic nude male mice 4 weeks of age by subcutaneously injecting 2.0×10⁶ cells suspended in 100 μl Matrigel (BD Biosciences) into both flanks. When palpable tumors established at approximately 5 mm diameter (66 mm³), intratumoral treatment with siPORT amine transfection reagent (Ambion) complexed miR-644a (3.15 μg of synthetic mimics every 3 days injection) was done in treatment groups (n=6 animals). Mice in Enzalutamide treatment group was fed with the drug through oral gavage every 3 days (10 mg/kg/day). Anti-tumor effects of miR-644a with or without Enzalutamide treatment was assessed thrice a week by measuring tumor volume. The tumor growth was monitored for 21 days from the start of treatment. Tumors and organs were harvested to understand xenograft metabolism. All the animal care was done by institutional guidelines.

Gene Enrichment analysis. The Prostate Cancer patient gene expression data was obtained from COSMIC Cancer Browser database from adenocarcinoma samples with high-level amplifications (n=498). The samples were collated, and the genes are displaying overexpression pattern were selected for further analysis. The miR-644a target pool was selected from prediction algorithms including Targetscan 6.2, miRanda, miRtarget2, and RNAhybrid algorithms. Next, the target pool was enriched using g:Profiler software (http://biit.cs.ut.ee/gprofiler/index.cgi) using the options significant only and ordered query with a p-value <0.05 and Benjamini-Hochberg FDR significance threshold. The results were visualized using GeneMania plugin for Cytoscape 3.1 (http://www.genemania.org/). Further analysis was performed using Reactome (http://www.reactome.org/) and EnrichR (http.//amp.pharm.mssm.edu/Enrichr/) pathway databases using p-value <0.05.

Testing relationships between ITCH CNV and miR-644a target genes in prostate cancer. All analyses, unless otherwise indicated, were undertaken using the R platform for statistical computing (R version 3.3.1 (2016-06-21), Platform: x86_64-apple-darwin13.4.0 (64-bit), Running under: OS X 10.11.6 (El Capitan) (R Core Team (2013). R: A language and environment for statistical computing. R Foundation for Statistical Computing, Vienna, Austria. ISBN 3-90005107-0, URL http://www.R-project.org/), and a range of library packages were implemented in Bioconductor as indicated.

Expression levels of the miR-644a genes were measured in the PRAD cohort. For the NEPC cohort, the cgdsr tool in was used to gain ITCH CNA and miR-644a target gene expression. In the first instance, the cross-correlation of ITCH CNA and miR-644a target expression was undertaken in R corrplot. In the PRAD cohort, the TCGA_PRAD_HiSeqV2 RNA-Seq data and associated clinical data were downloaded from UCSC and tumor:normal Z-scores calculated to yield the tumor expression of all detectable genes, in at least 80%, relative to normal tissue. The tumor-associated expression alterations of all detectable miR-644a target genes were measured (Z-scores), and genefilter was used to select for genes that were commonly and significantly altered. Specifically, a threshold for filtering was taken that selected genes that were altered by more than 2 Z scores in 30% of tumors. The expression of genes was used to cluster tumors and visualized with heatmap. The association of patient cluster membership and clinical outcome (either categorical data or continuous data that was categorized) was then tested using a Chi-squared test and regression models constructed using survival (survival).

As disclosed and suggested herein, the scope of the general inventive concepts are not intended to be limited to the particular exemplary embodiments shown and described herein. From the disclosure given, those skilled in the art will not only understand the general inventive concepts and their attendant advantages but will also find apparent various changes and modifications to the methods and systems disclosed. It is sought, therefore, to cover all such changes and modifications as fall within the spirit and scope of the general inventive concepts, as described and suggested herein, and any equivalents thereof.

What is claimed is:

1. A method for the treatment of prostate cancer in an individual, the method comprising administering a composition to increase a level of miR-644a in the individual.

2. The method of claim 1, wherein increasing the level of miR-644a in the individual modulates expression of Bcl-x1 and/or Bcl-2.

3. The method of claim 1, wherein the individual exhibits one or more symptoms of castration-resistant prostate cancer.

4. The method of claim 3, wherein the individual has been diagnosed with castration-resistant prostate cancer.

5. The method of claim 1, wherein the individual has undergone androgen deprivation therapy.

6. The method of claim 2, wherein modulating expression of Bcl-x1 and/or Bcl-2 results in a reduction in tumor growth.

7. The method of claim 1, wherein the composition increases a level of miR149-5p.

8. The method of claim 1 further comprising administration of a pharmaceutical prostate cancer therapy.

9. A method of reducing or treating prostate cancer therapeutic resistance the method comprising:
administering a composition to increase a level of miR-644a in the individual.

10. The method of claim 9, wherein increasing the level of miR-644a in the individual modulates expression of Bcl-x1 and/or Bcl-2.

11. The method of claim 9, wherein the individual exhibits one or more symptoms of castration-resistant prostate cancer.

12. The method of claim 9, wherein the individual has been diagnosed with castration-resistant prostate cancer.

13. The method of claim 9, wherein the individual has undergone androgen deprivation therapy.

14. The method of claim 10, wherein modulating expression of Bcl-x1 and/or Bcl-2 results in a reduction in tumor growth.

15. The method of claim 9, wherein the composition increases a level of miR149-5p.

16. A composition for the treatment of prostate cancer comprising a vector for increasing a level of miR-644a in an individual.

17. The composition of claim 16, wherein the vector also increases a level of miR-149-5p.
